(19) United States
(12) Patent Application Publication
(10) Pub. No.: US 2023/0057861 A1
(43) Pub. Date: Feb. 23, 2023

(54) USE OF CORYDALIS SAXICOLA BUNTING AND FORMULATION THEREOF IN PREPARATION OF DRUG FOR TREATING NON-ALCOHOLIC FATTY LIVER DISEASES

(71) Applicants: NANJING ZHONGSHAN PHARMACEUTICAL CO., LTD., Nanjing, Jiangsu (CN); JIANGSU HONGDIAN RESEARCH INSTITUTE OF TRADITIONAL CHINESE MEDICINE INDUSTRY CO., LTD., Nanjing, Jiangsu (CN)

(72) Inventors: Jun CHENG, Nanjing (CN); Fang HUANG, Nanjing (CN); Kaijun ZHAO, Nanjing (CN)

(73) Assignees: NANJING ZHONGSHAN PHARMACEUTICAL CO., LTD., Nanjing, Jiangsu (CN); JIANGSU HONGDIAN RESEARCH INSTITUTE OF TRADITIONAL CHINESE MEDICINE INDUSTRY CO., LTD., Nanjing, Jiangsu (CN)

(21) Appl. No.: 17/797,305

(22) PCT Filed: Feb. 23, 2021
(86) PCT No.: PCT/CN2021/077410

§ 371 Date: Aug. 3, 2022

(30) Foreign Application Priority Data
Mar. 13, 2020 (CN) 202010175797.4

(51) Int. Cl. A61K 36/66; A61K 9/48; A61P 1/16

(52) U.S. Cl. CPC A61K 36/66; A61K 9/48; A61P 1/16; A61K 2236/39; A61K 2236/51

(57) ABSTRACT

Provided is use of Corydalis saxicola Bunting and a Corydalis saxicola Bunting total alkaloid capsule in the preparation of drugs for treating non-alcoholic fatty liver diseases, non-alcoholic steato-hepatitis, drug induced liver injury, and chemical liver injury.

### TECHNICAL FIELD

The present invention relates to a new use of Corydalis saxicola Bunting and a Corydalis saxicola Bunting total alkaloid capsule, and in particular, to use of Corydalis saxicola Bunting and a Corydalis saxicola Bunting total alkaloid capsule in treatment of non-alcoholic fatty liver diseases and non-alcoholic steato-hepatitis, which belongs to the technical field of medicine.

### BACKGROUND

Non-alcoholic fatty liver disease (NAFLD) is defined as hepatic steatosis confirmed by imaging and liver histology, excluding other causes of hepatic steatosis, such as heavy alcohol consumption, long-term use of adipogenic drugs, or monogenic disorders. According to its clinical manifestations, it mainly includes hepatic steatosis, non-alcoholic steato-hepatitis (NASH), and cirrhosis. The pathogenesis of NAFLD is very complex, and there is currently no recognized treatment with effective drugs. Therefore, at present, there is a need for continuous efforts on the development of effective drugs on the basis of the research on the pathogenesis.

In addition, with the improvement of people's living standard in China and the increasing use of traditional Chinese medicine and health products, the incidence of drug-induced hepatotoxicity is on the rise. Drug-induced hepatotoxicity, also referred to as drug induced liver injury (DILI), is the second largest type of non-infectious liver disease in China. The drug-induced hepatotoxicity is now a major cause of acute liver failure. Drugs withdrawn from the market due to hepatotoxicity have cost billions of dollars over the past few decades.

The main component of Corydalis saxicola Bunting is Corydalis saxicola Bunting total alkaloid (CSBTA). Corydalis saxicola Bunting has the effects of clearing away heat and removing toxic substances, removing dampness, relieving pain and stopping bleeding. It is commonly used for hepatitis, erosion of the mouth and tongue, burning eyes, nebula, dysentery, diarrhea, abdominal pain, and bleeding hemorrhoids. A CSBTA capsule has completed clinical phase II, and is under clinical phase III by Nanjing Zhongshan Pharmacy Co., Ltd.

### SUMMARY

Objective of the invention: The purpose of the invention is to overcome the shortcomings of the prior art, and to develop new clinical efficacy and expand its clinical indications on the basis of the existing efficacy of corydalis saxicola and its corydalis saxicola total alkaloids capsules through a large number of experiments and screening.

Technical solutions: To achieve the foregoing objective, the present invention adopts the following technical solutions:

Use of Corydalis saxicola Bunting in the preparation of a drug for treating non-alcoholic fatty liver diseases.

Use of Corydalis saxicola Bunting in the preparation of a drug for treating non-alcoholic steato-hepatitis.

Use of Corydalis saxicola Bunting in the preparation of a drug for treating non-alcoholic fatty liver diseases combined with drug induced liver injury.

Use of Corydalis saxicola Bunting in the preparation of a drug for treating non-alcoholic fatty liver diseases combined with chemical liver injury.

Use of a Corydalis saxicola Bunting total alkaloid capsule in the preparation of a drug for treating non-alcoholic fatty liver diseases.

Use of a Corydalis saxicola Bunting total alkaloid capsule in the preparation of a drug for treating non-alcoholic steato-hepatitis.

Use of a Corydalis saxicola Bunting total alkaloid capsule in the preparation of a drug for treating non-alcoholic fatty liver diseases combined with drug induced liver injury.

Use of a Corydalis saxicola Bunting total alkaloid capsule in the preparation of a drug for treating non-alcoholic fatty liver diseases combined with chemical liver injury.

In the present invention, the Corydalis saxicola Bunting total alkaloid is prepared by a method including the following steps:

(1) 30 kg of Corydalis saxicola Bunting slices were soaked into an ethanol having a concentration of 75% at an amount of 10 times the volume of the Corydalis saxicola Bunting slices for 1 h.

(2) Heating under reflux was carried out for 3 times of extraction, each for 2 h, and extracts were combined.

(3) The ethanol was fully recovered, and the extract was concentrated to obtain a concentrate of 1:1.3 (Weight of Corydalis saxicola Bunting slices: volume of concentrated solution).

(4) 1% hydrochloric acid of 4 times the amount of raw materials (Corydalis saxicola Bunting slices) was added into the concentrate, and fed into an acid-resistant multifunctional extraction tank and heated under reflux for 1 h of extraction to obtain an extract.

(5) A pH value of the extract was adjusted and maintained to 8 with 40% sodium hydroxide, then concentrated to obtain a concentrate with a relative density of 1.06-1.08 (60° C.), the concentrate was refrigerated, standing for 48 h, and then filtered, to obtain a precipitate; the precipitate was heated under reflux with 1% hydrochloric acid of 16 times the amount of the precipitate (W/V) for 1 h of extraction, and filtered while hot to obtain a filtrate.

(6) A pH value of the filtrate was adjusted and maintained to 6-8 with 40% sodium hydroxide, and the filtrate was then concentrated to obtain a concentrate with a relative density of 1.06-1.08 (60° C.), the concentrate was refrigerated, standing for 48 h, and then filtered to obtain a precipitate.

(7) The precipitate was dried under reduced pressure (60° C., 0.08 MPa) to obtain a Corydalis saxicola Bunting total alkaloid extract.

### Technical Effects of the Present Invention

In the present invention, through a large number of experimental screening, a new clinical efficacy of Corydalis saxicola Bunting and a Corydalis saxicola Bunting total alkaloid (CSBTA) capsule prepared therefrom is developed on the basis of their existing efficacy. It was found through experiments that Corydalis saxicola Bunting and a CSBTA capsule can not only effectively reduce the synthesis of fatty acids in cells, but also reduce the levels of TG, TC, LDL cholesterol, HDL cholesterol, and free fatty acids that are induced by HFD. In addition, Anatomical experiments shown that CSBTA can reduce the weight of liver while pathological sections results shown that it can well protects liver from injury. Moreover, it was found through clinical experiments that CSBTA can significantly improve the biochemical indicators and imaging evaluation results of patients with fatty liver. In addition, in the present invention, it was shown from the results of the drug induced liver injury experiment that the CSBTA capsule can significantly alleviate drug induced liver injury and has the effect of preventing liver injury.

Moreover, in the present invention, it was shown from the results of the drug induced liver injury experiment that the CSBTA capsule can significantly alleviate chemical and drug induced liver injury and has the effect of preventing liver injury. Through the experiments of the present invention, the clinical indications of CSBTA can be expanded, providing an effective basis for its clinical promotion.

### BRIEF DESCRIPTION OF THE DRAWINGS

FIG. 1 is a histogram of a cytotoxicity experiment.

FIG. 2 is a histogram of a liver weight index of mice in each group.

FIG. 3 is a histogram of blood glucose changes in mice in each group.

FIG. 4 shows histograms of lipid distribution of mice in each group.

FIG. 5 shows the results of HE staining.

FIG. 6 shows the results of Oil Red O staining.

In the foregoing figures, C represents a model group, B represents a blank group, L represents a Corydalis saxicola Bunting total alkaloid (25 mg/kg) group, H represents a Corydalis saxicola Bunting total alkaloid (100 mg/kg) group, and MET represents a metformin (200 mg/kg) group. * indicates that, compared with the model group, it is statistically significant P<0.05. **** indicates that, compared with the model group, it is statistically significant P<0.01.

### DETAILED DESCRIPTION

The present invention is further described below by using examples, but the present invention is not limited to the scope of the described examples. After reading the present invention, various insubstantial modifications to the present invention by a person skilled in the art shall fall within the scope defined by the appended claims of the present application.

### Example 1 Preparation of Corydalis saxicola Bunting Total Alkaloid (CSBTA)

(1) 30 kg of Corydalis saxicola Bunting slices were soaked into an ethanol having a concentration of 75% at an amount of 10 times the volume of the Corydalis saxicola Bunting slices for 1 h.

(2) Heating under reflux was carried out for 3 times of extraction, each for 2 h, and extracts were combined.

(3) The ethanol was fully recovered, and the extract was concentrated to obtain a concentrate of 1:1.3 (Weight of Corydalis saxicola Bunting slices: volume of concentrated solution).

(4) 1% hydrochloric acid of 4 times the amount of raw materials (Corydalis saxicola Bunting slices) was added into the concentrate, and fed into an acid-resistant multifunctional extraction tank and heated under reflux for 1 h of extraction to obtain an extract.

(5) A pH value of the extract was adjusted and maintained to 8 with 40% sodium hydroxide, the extract was then concentrated to obtain a concentrate with a relative density of 1.06-1.08 (60° C.), the concentrate was refrigerated, standing for 48 h, and then filtered, to obtain a precipitate; the precipitate was heated under reflux with 1% hydrochloric acid of 16 times the amount of the precipitate (W/V) for 1 h of extraction, and filtered while hot to obtain a filtrate.

(6) A pH value of the filtrate was adjusted and maintained to 6-8 with 40% sodium hydroxide, and the filtrate was then concentrated to obtain a concentrate with a relative density of 1.06-1.08 (60° C.), the concentrate was refrigerated, standing for 48 h, and then filtered to obtain a precipitate.

(7) The precipitate was dried under reduced pressure (60° C., 0.08 MPa) to obtain a CSBTA extract.

According to clinical needs, 125 g of soluble starch was added into 75 g of CSBTA, mixed uniformly, granulated by adding a proper amount of 85% ethanol, dried, broken, and put into a capsule, to obtain a CSBTA capsule.

### Example 2 Observation of Therapeutic Effect of CSBTA Capsule in Cell Experiment

1. Experimental drug: Corydalis saxicola Bunting total alkaloid (CSBTA) prepared in Example 1.

2. Cytotoxicity experiment 1.1 Experimental instrument: MULTISKAN Sky full-wavelength microplate reader (Thermo Scientific, US).

1.2 Reagents: MTT Cell Proliferation and Cytotoxicity Assay Kit (cat #KGA321, Jiangsu KeyGEN BioTECH Corp., Ltd.); CSBTA prepared in Example 1.

1.3 Experimental method: Cells were added in a 96-well plate at 100 μL/well (about 1×10⁴ cells/well), and cultured in a cell incubator at 37° C. under 5% CO₂ for 24 h. The culture medium in all wells was pipetted out. A CSBTA (100 μg/mL) solution was prepared with a blank medium without serum, and was diluted serially to seven concentrations of 50, 25, 12.5, etc. of CSBTA in a two-fold gradient. The blank medium and the media of different concentrations were added into the 96-well plate, and cultured in a cell incubator at 37° C. under 5% CO₂ and 100% humidity for 24 h. 5×MTT was diluted to 1×MTT with a dilution buffer. 50 μL of 1×MTT was added into each well and cultured at 37° C. for 4 h to reduce MTT to formazan. A supernatant was pipetted out. 150 μL of DMSO was added into each well to dissolve formazan and shaken up by a plate shaker. An optical density at a wavelength of 490 nm in each well was measured by a microplate reader.

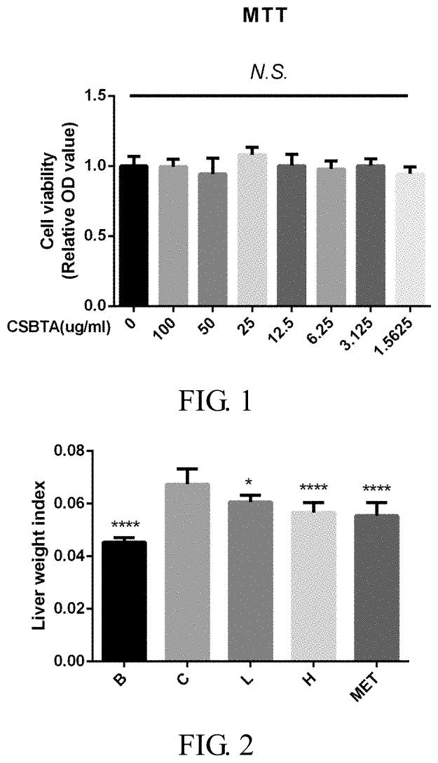

1.4 Experimental results: To determine the optimal dosage of the CSBTA capsule, its toxicity to cells was tested in seven gradients. As shown in the test results in FIG. 1, it was found that the CSBTA capsule has no significant toxicity to cells.

### Example 3: Effect of Corydalis saxicola Bunting Total Alkaloid (CSBTA) on Reducing Blood Lipid in Hyperlipidemia Model Rats

1. Experimental modeling

1.1 Experimental animal: Four-week-old male C57BL/6J mice (about 19 g) were purchased from the Center for Comparative Medicine of Yangzhou University.

1.2 Feed: 60% high-fat chow, fructose, normal chowwere purchased from Nantong Trophic Animal Feed High-Tech Co., Ltd.

1.3. Reagent: CSBTA prepared in Example 1.

1.4 Experimental method: After adaptive feeding for one week, 35 four-week-old male C57BL/6 mice were randomized into 2 initial groups, fed with a normal chow or a high-fat (60% fat) high-sugar (20% fructose) chow. After feeding for 10 weeks, the mice were randomized into a model group, a CSBTA (25 mg/kg) group, a CSBTA (100 mg/kg) group, a metformin (200 mg/kg) group (n=7), and a blank group (with an equal volume of 0.5% methylcellulose (the Chinese Pharmacopoeia, Beijing)). During modeling, the mice were given ad libitum access to food and water at room temperature of 23-25° C. in an animal laboratory. The proper temperature and the 12-hour day-night cycle were maintained. From week 11, the mice in each group were dosed by gavage for 5 weeks, and its weight was measured once a week.

1.5 Experimental results

Liver weight index of mice

As shown in FIG. 2, The liver weight of the model group increased, swollen and turned brown, which was significantly improved in the administration group and the positive drug group. 2. Changes of blood glucose in mice

2.1 Experimental instrument: Yuwell blood glucose meter, blood glucose test strip.

2.2 Reagent: Glucose (sigma, US).

2.3 Experimental method: After the administration of item 1.4, the OGTT was carried out on the mice to observe the function of islet beta cell and the ability of body to regulate blood glucose. The mice were fasted for 12 h after 8 pm. The fasting blood glucose (FBG) was measured in the next day. A glucose solution was given to the mice in each group by gavage (2 g/kg). The blood glucose in mice was measured at 15 min, 30 min, 60 min, and 120 min after the gavage of glucose. The area under the curve (AUC) was calculated.

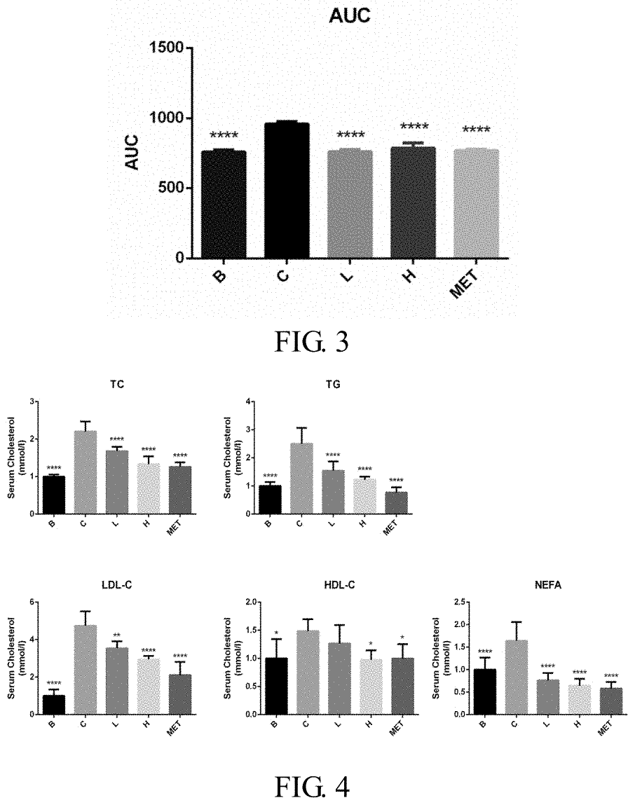

2.4 Experimental results: It can be seen from the AUC in FIG. 3 that, compared with the mice in the model group, the CSBTA had beneficial effect on glucose tolerance. In addition, the loss of FBG caused by HFD was also largely restored by the CSBTA treatment.

3. Changes of blood lipid in mice

3.1 Experimental instrument: MULTISKAN Sky full-wavelength microplate reader (Thermo Scientific, US).

3.2 Reagents: TC Assay Kit (A111-1-1, Nanjing Jiancheng Biotechnology Co., Ltd.), TG Assay Kit (A110-1-1, Nanjing Jiancheng Biotechnology Co., Ltd.), HDL-C Assay Kit (A112-1-1, Nanjing Jiancheng Biotechnology Co., Ltd.), LDL-C Assay Kit (A113-1-1, Nanjing Jiancheng Biotechnology Co., Ltd.), and NEFA Assay Kit (A042-2-1, Nanjing Jiancheng Biotechnology Co., Ltd.).

3.3 Experimental method: The mice were fasted overnight. Blood samples were collected from ophthalmic veins and centrifuged at 4° C. at 3000 rpm for 10 min. The plasma was collected and stored at −80° C. for use. The kits were applied according to the manufacturer's instructions.

3.4 Experimental results: FIG. 4 shows the effect of CSBTA on the metabolic characteristics of HFD-fed mice, and the lipid distribution were measured, including the contents of TG, TC, LDL cholesterol, HDL cholesterol, and free fatty acids. CSBTA can significantly reduce the levels of TG, TC, LDL cholesterol, HDL cholesterol, and free fatty acids that are induced by HFD.

4. Histopathological analysis

4.1 HE staining

4.1.1 Experimental method: Fresh liver tissue was collected and fixed in 4% neutral formalin buffer for 24 h, and then dehydrated. The tissue blocks were trimmed, processed, and embedded in paraffin. The tissue in glass slides were cut to the thickness of 5 μm and treated with HE staining.

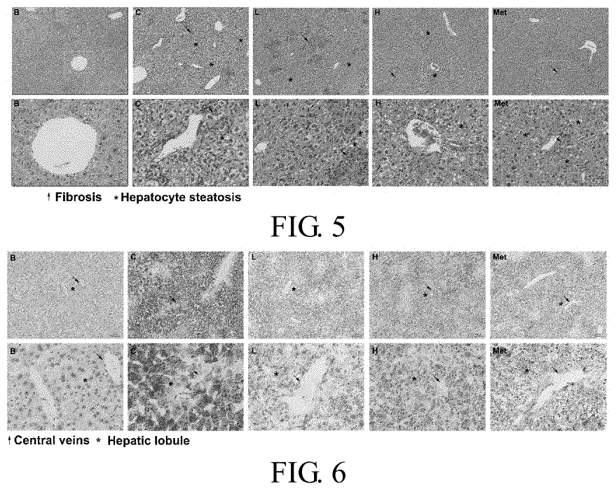

4.1.2 Experimental results: As shown in panel B in FIG. 5, the profile of the central vein in normal liver tissue is clear, the hepatic cords are arranged radially along the central vein, the cytoplasm of the liver cells is loose, the central vein is shown in the yellow frame, and there is no significant inflammatory cell infiltration in the tissue. As shown in panel C, the histopathological analysis on the liver of HFD mice with HE staining shows hepatocyte swelling, increased steatosis, sparse hepatocyte cytoplasm, and bundles of intermediate filaments. The foregoing phenomena are significantly relieved upon dosing with Corydalis saxicola Bunting total alkaloid (25 mg/kg), Corydalis saxicola Bunting total alkaloid (100 mg/kg), and metformin.

4.2 Oil Red O staining

4.2.1 Experimental method: For Oil Red O staining, frozen sections (6 μm thick) of the liver were incubated in 10% formalin at room temperature for 30 min, and then stained with a fresh Oil Red O working solution for 20 min. After washed with water, the sections were counterstained with hematoxylin dye for 1 min and placed under a microscope to observe lipid deposition.

4.2.2 Experimental results: As shown in panel C in FIG. 6, there is significant lipid droplet aggregation around cells, and this phenomenon is significantly relieved in the low-dose CSBTA (25 mg/kg) group and the positive dosage group, and is more significantly relieved in the high-dose CSBTA (100 mg/kg) group.

4.3 Masson staining

4.3.1 Experimental method: The staining was carried out according to the manufacturer's instructions.

4.3.2 Experimental results: In the blank group, a small amount of collagen fibers can be seen around the vessel wall, within the normal range. In the model group, a large amount of collagen fibers in the liver tissue are deposited with blue color, and extend outward from the portal area, and the fibrous strands are thick and stained darkly, indicating that there are many collagen fibers wrapped. The foregoing phenomena are significantly relieved in the low-dose CSBTA (25 mg/kg) group, and almost disappear in the high-dose CSBTA (100 mg/kg) group and the positive dosage group.

The foregoing descriptions are exemplary implementations of the present invention. It should be noted that a person of ordinary skill in the art may make some improvements and modifications without departing from the principle of the present invention and the improvements and modifications shall fall within the protection scope of the present invention.

1. A method for treating a non-alcoholic fatty liver disease, comprising administering a drug comprising Corydalis saxicola Bunting to a subject having non-alcoholic fatty liver disease.

2. The method of claim 1, wherein the subject has non-alcoholic steato-hepatitis.

3. The method of claim 1, wherein the subject has non-alcoholic fatty liver disease combined with drug induced liver injury.

4. The method of claim 1, wherein the subject has non-alcoholic fatty liver disease combined with chemical liver injury.

5. A method for treating a non-alcoholic fatty liver disease, comprising administering a Corydalis saxicola Bunting total alkaloid capsule to a subject having non-alcoholic fatty liver disease.

6. The method of claim 5, wherein the subject has non-alcoholic steato-hepatitis.

7. The method of claim 5, wherein the subject has non-alcoholic fatty liver disease combined with drug induced liver injury.

8. The method of claim 5, wherein the subject has non-alcoholic fatty liver disease combined with chemical liver injury.

9. The method of claim 5, wherein the Corydalis saxicola Bunting total alkaloid capsule is prepared by a method comprising:
(1) soaking Corydalis saxicola Bunting slices into an ethanol having a concentration of 50-75% at an amount of 8-10 times the volume of the Corydalis saxicola Bunting slices for 0.5-1 h;
(2) heating under reflux for 1 to 3 times of extraction, each for 0.5-2 h, and combining extracts;
(3) fully recovering the ethanol, and concentrating to obtain a concentrate;
(4) adding hydrochloric acid into the concentrate, and feeding it into an acid-resistant multifunctional extraction tank and heating it under reflux for extraction to obtain an extract;
(5) adjusting and maintaining a pH value of the extract with sodium hydroxide, concentrating the extract to obtain a concentrate, refrigerating the concentrate, standing, and filtering, to obtain a precipitate; heating the precipitate under reflux with hydrochloric acid for extraction, and filtering it while hot to obtain a filtrate;
(6) adjusting and maintaining a pH value of the filtrate to 6-8 with sodium hydroxide, concentrating, refrigerating, standing, and filtering it to obtain a precipitate; and
(7) drying the precipitate under reduced pressure to obtain a Corydalis saxicola Bunting total alkaloid extract, and preparing it with a pharmaceutically acceptable carrier into a capsule.
